(19) United States
(12) Patent Application Publication
(10) Pub. No.: US 2023/0417433 A1
(43) Pub. Date: Dec. 28, 2023

(54) SYSTEM AND METHOD FOR OPERATING CLIMATE CONTROL TO REDUCE VIRUS TRANSMISSION

(71) Applicant: Bergstrom, Inc., Rockford, IL (US)

(72) Inventor: Brett Sean Connell, Rockford, IL (US)

(21) Appl. No.: 18/329,507

(22) Filed: Jun. 5, 2023

Related U.S. Application Data
(60) Provisional application No. 63/350,843, filed on Jun. 9, 2022.

(51) Int. Cl. F24F 11/00

(52) U.S. Cl. CPC F24F 11/0008; F24F 2110/64

(57) ABSTRACT

The method for reducing virus transmission in an enclosed space includes providing a minimum humidity ratio threshold at which transmission of a particular virus is decreased. The method also includes determining a humidity ratio of an enclosed space suitable for receiving a living being. The method also includes, in response to determining that the humidity ratio is less than the minimum humidity ratio threshold, increasing the humidity ratio until the humidity ratio in the enclosed space is at least equal to the minimum humidity ratio threshold.

### RELATED APPLICATIONS

This application claims priority to U.S. Provisional Patent Application No. 63/350,843, filed Jun. 9, 2022, entitled “System And Method For Operating Climate Control To Reduce Virus Transmission,” which is hereby incorporated by reference in their entirety.

### TECHNICAL FIELD

The disclosed implementations relate generally to heating, ventilation, and cooling (HVAC) climate control systems for vehicles or buildings. In particular, the disclosed implementations relate to systems and methods for operating HVAC climate control systems to reduce virus transmission in an enclosed space.

### BACKGROUND

It has been found that for many viruses, transmission rates decrease when humidity increases. Influenza, for example, transmits at lower rates in higher humidity. SARS-Cov-2 (i.e., the virus that causes COVID-19) may also transmit at lower rates in higher humidity. This is at least partially the result of virus particles decaying more quickly in more humid climates.

Of course, there are a number of ways to indicate humidity, including relative humidity, absolute humidity, and humidity ratio. Depending on the virus, a function describing transmission rates at different humidities may track more cleanly onto one measure of humidity than another. For example, hypothetical virus X may be more sensitive to relative humidity; whereas virus Y may be more sensitive to humidity ratio.

It is desirable to provide a method of monitoring and adjusting indoor climate, including indoor humidity, to reduce or eliminate virus transmission.

### SUMMARY

The following provides a description of systems and methods for operating a climate control system to reduce virus transmission. Operating a climate control system to adjust the humidity of an enclosed space provides a reliable, non-invasive method of decreasing virus transmission rates in an enclosed space.

Some implementations provide a method for reducing virus transmission in a space. This method includes providing a minimum humidity ratio threshold at which transmission of a particular virus is decreased, determining a humidity ratio of an enclosed space suitable for receiving a living being, and, in response to determining that the humidity ratio is less than the minimum humidity ratio threshold, increasing the humidity ratio until the humidity ratio in the enclosed space is at least equal to the minimum humidity ratio threshold. In some implementations, the minimum humidity ratio threshold includes a minimum humidity ratio at which a half-life of a virus is decreased. In some implementations, the minimum humidity ratio threshold is within a comfort zone. In some implementations, the comfort zone includes an ASHRAE Standard 55 comfort zone, an EN 15251 comfort zone, or a Givoni-Milne Bioclimatic Chart comfort zone. In some implementations, determining the humidity ratio includes detecting the humidity ratio with a sensor.

In some implementations, determining the humidity ratio includes detecting at least two of the following metrics: a dry-bulb temperature of the space, a wet-bulb temperature of the space, an absolute humidity of the space, a relative humidity of the space, an air pressure of the space, a vapor pressure of the space, a volume of air in the space, and an enthalpy of the air in the space, and calculating the humidity ratio based on the at least two detected metrics. In some implementations, increasing the humidity ratio includes operating a humidifier, disabling a dehumidifier, introducing air outside the space into the space, maintaining a temperature of a coil above a dew point of the enclosed space, or disabling a compressor. In some implementations, the method includes determining whether a living being approached, entered, or occupied the enclosed space.

In some implementations, the method includes maintaining the humidity ratio after determining that the humidity ratio is at least equal to the minimum humidity ratio threshold. In some implementations, the method includes maintaining, after determining the humidity ratio is at least equal to a maximum humidity ratio threshold, the humidity ratio between the minimum humidity ratio threshold and the maximum humidity ratio threshold. In some implementations, the maximum humidity ratio threshold is within a comfort zone. In some implementations, maintaining the humidity ratio includes maintaining the humidity ratio for a predetermined amount time. In some implementations, the predetermined amount of time is based on the half-life of a virus at the minimum humidity ratio threshold. In some implementations, the method includes decreasing the humidity ratio after determining the humidity ratio is greater than a maximum humidity ratio threshold.

Some implementations provide a system for reducing virus transmission in a space, including one or more sensors, and a controller electrically coupled to the one or more sensors, wherein the controller is configured to perform any of the aforementioned methods. Some implementations include a non-transitory, computer-readable medium comprising instructions that, when executed by one or more processors, cause the one or more processors to perform any of the aforementioned methods.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the drawings.

FIG. 1 is a schematic representation of an enclosed space, in accordance with some implementations.

FIG. 2 is a schematic representation of an HVAC system, in accordance with some implementations.

FIG. 3 is a block diagram illustrating a controller, in accordance with some implementations.

FIG. 4 is a flow chart of a method for reducing virus transmission in an enclosed space, in accordance with some implementations.

FIGS. 5A-5E are example psychrometric charts, some of which include notations that demonstrate certain aspects of the present invention.

### DETAILED DESCRIPTION

Reference will now be made in detail to implementations, examples of which are illustrated in the accompanying drawings. In the following detailed description, numerous specific details are set forth in order to provide a thorough understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

Many modifications and variations of this disclosure can be made without departing from its spirit and scope, as will be apparent to those skilled in the art. The specific implementations described herein are offered by way of example only, and the disclosure is to be limited only by the terms of the appended claims, along with the full scope of equivalents to which such claims are entitled.

Implementations of the present disclosure are described in the context of refrigerant systems for controlling the climate in an enclosed space as well as reducing the probability of virus transmission within the use enclosed space. The refrigerant systems disclosed herein can control the humidity within an enclosed space to decrease the transmission of a particular virus while keeping the enclosed space comfortable or suitable for a living being to stay for prolonged periods of time.

As used herein, a “refrigerant” is a fluid adapted to undergo phase transitions between liquid and gas during operation of a corresponding refrigerant system. For example, the refrigerant has a liquid-to-gas transition point below a target operating temperature of the refrigerant system. In various implementations, the refrigerant may be a class 1, class 2, or class 3 refrigerant.

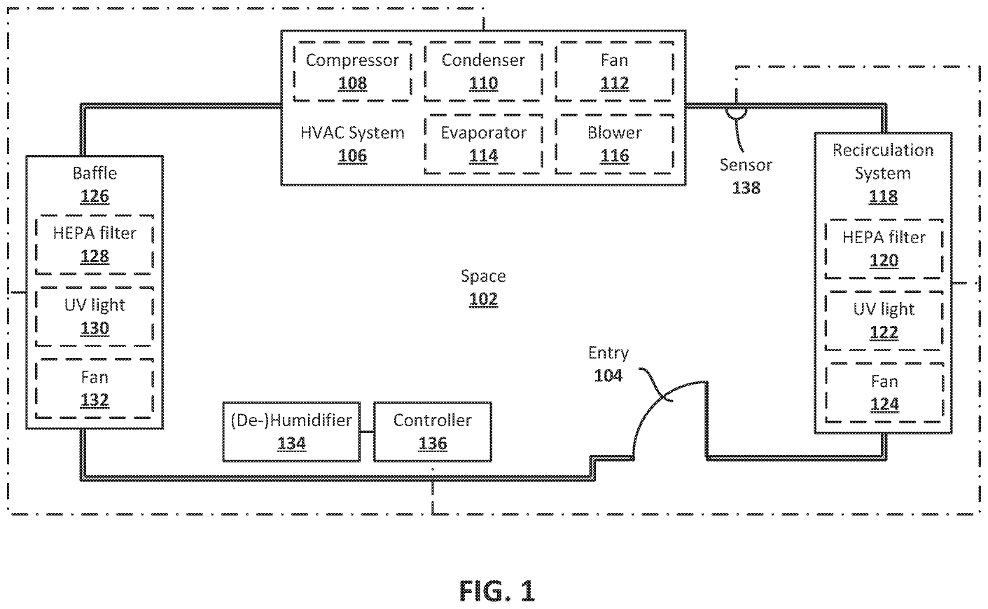

FIG. 1 is a schematic representation 100 of an enclosed space 102. In some embodiments, the enclosed space 102 is an interior portion of a vehicle, such as a cabin of a truck, an airplane, a ship, or a train. In some embodiments, the enclosed space 102 is an interior portion of a building, such a room in a home, a classroom in a school, an auditorium in a theatre, or the floor of an office building. In some embodiments, the enclosed space 102 is the entire interior of a vehicle (e.g., each cabin of a train) or a building (e.g., each room in a home).

In some embodiments, the enclosed space 102 has an entry 104 to the space. In some embodiments, the entry 104 is a door, such as a hinged door, a revolving door, or a sliding door. Alternatively, the entry 104 is a doorway, a window, or any other entrance or exit through which a living being can access the enclosed space 102. In some embodiments, the enclosed space 102 includes multiple such entries 104. For example, a home with a front door, a back door, a pet door, a garage door, and one or more windows constitutes an enclosed space with one or more entries.

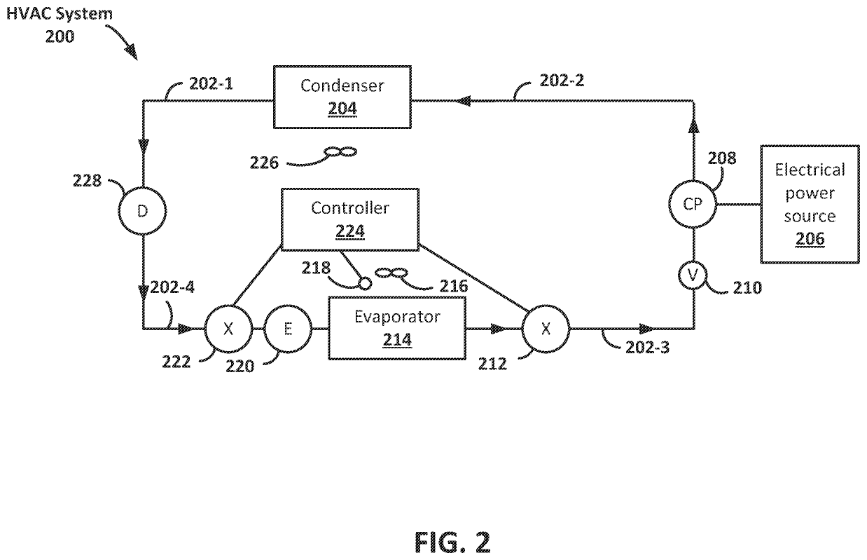

In some embodiments, the enclosed space 102 includes a heating, ventilation, and air-conditioning (HVAC) system 106. In some embodiments, the HVAC system 106 includes a compressor 108, a condenser 110, a fan 112, an evaporator 114, and a blower 116. It should be appreciated that the HVAC system can include more or fewer components. For details on the operation of an example HVAC unit, see FIG. 2 and the related description.

In some embodiments, the enclosed space 102 includes a recirculation system 118. In some embodiments, the recirculation system 118 includes a high-efficiency particulate air (HEPA) filter 120 or an ultraviolet (UV) light 122. In some embodiments, the recirculation system 118 uses the HEPA filter 120 and/or the UV light 122 to filter air from the enclosed space 102 and kill bacteria in the air. In some embodiments, the recirculation system 118 includes one or more fans 124. In some embodiments, the recirculation system uses the one or more fans 124 to pull air from the enclosed space 102 into the recirculation system 118, through the HEPA filter and/or past the UV light, and then back into the enclosed space 102. In some embodiments, the recirculation system 118 uses the one or more fans 124 to circulate air in the enclosed space 102. In some embodiments, the recirculation system is external to the enclosed space. For example, in some embodiments, the enclosed space is a room in a building and the recirculation system is part of the ventilation of the building—external to the room but fluidly coupled to it.

In some embodiments, the enclosed space 102 includes a baffle 126. In some embodiments, the baffle 126 includes a HEPA filter 128 and/or a UV light 130. Like the recirculation system 118, in some embodiments, the baffle 126 uses the HEPA filter 128 to filter air from the enclosed space 102 and/or the UV light 130 to kill any bacteria/viruses in the air. In some embodiments, the baffle 126 also includes one or more fans 132. In some embodiments, the baffle 126 uses the one or more fans 132 to pull air from the enclosed space 102 out of the enclosed space 102. In some embodiments, the baffle 126 uses the one or more fans 132 to pull air from outside the enclosed space 102 into the enclosed space 102. For example, if the air outside the enclosed space is more humid than the air inside the enclosed space, the fan can pull air from outside the enclosed space to increase the humidity in the enclosed space.

In some embodiments, the enclosed space 102 includes a humidifier/dehumidifier 134. In some embodiments, the humidifier/dehumidifier 134 is a humidifier (e.g., a cool mist humidifier, a warm mist humidifier, an ultrasonic humidifier, an evaporative humidifier, or a vaporizer). In some embodiments, the humidifier/dehumidifier 134 is a dehumidifier (e.g., a heat pump dehumidifier, a dehumidifying ventilator, or a desiccant dehumidifier). In some embodiments, the humidifier/dehumidifier 134 includes one or more devices capable of increasing and decreasing the humidity (e.g., the humidity ratio) of the enclosed space 102. In FIG. 1, the humidifier/dehumidifier 134 is depicted as separate from the HVAC system 106. However, in some embodiments, the humidifier/dehumidifier 134 is part of the HVAC system 106.

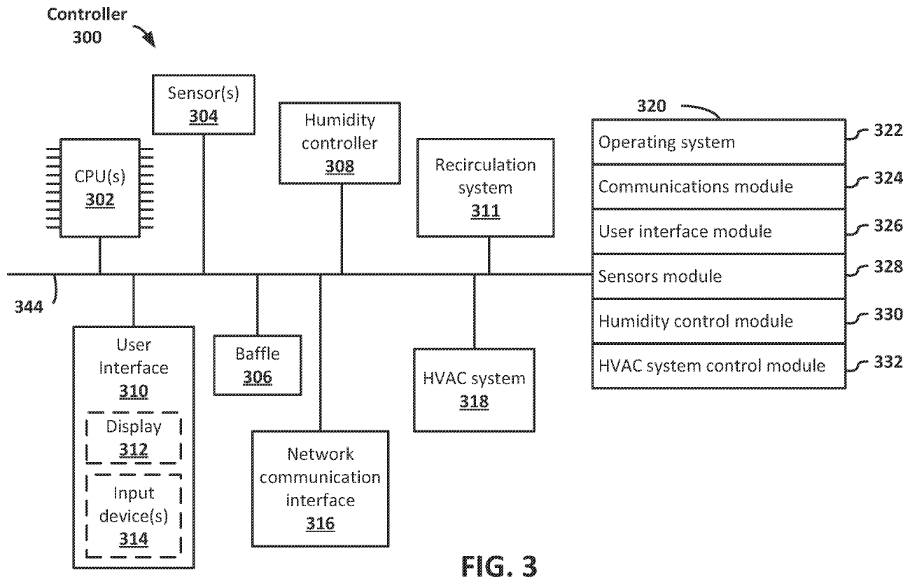

In some embodiments, the enclosed space 102 includes a controller 136. In some embodiments, the controller 136 is wirelessly or electrically coupled to one or more of the HVAC system 106, the recirculation system 118, the baffle 126, the humidifier/dehumidifier 134, and one or more sensors 138. In some embodiments, the controller operates automatically without any human intervention (e.g., performing the method 400 of flow chart). In some embodiments (e.g., with no sensors), a user may turn the system on manually. For details on the operation of an example controller, see FIG. 3 and the paragraphs relating to it.

In some embodiments, the enclosed space 102 includes the one or more sensors 138. In some embodiments, the one or more sensors 138 include a proximity sensor, a heat sensor, an optical sensor (e.g., a camera), or a motion sensor. In some embodiments, the one or more sensors 138 are configured to detect whether a living being entered the enclosed space 102. In some embodiments, the one or more sensors 138 include a dry-bulb thermometer, a wet-bulb thermometer, a capacitive humidity sensor, a resistive humidity sensor, a thermal humidity sensor, a barometer, or a pressure sensor. In some embodiments, the one or more sensors 138 detect the humidity (e.g., the absolute humidity, the relative humidity, or the humidity ratio) of the enclosed space 102, or metrics from which the humidity of the enclosed space 102 can be calculated.

FIG. 2 is a schematic representation of an HVAC system 200, such as the HVAC system 106 of FIG. 1, in accordance with some embodiments of the present invention. As depicted in FIG. 2, the HVAC system 200 includes a compressor 208, a condenser 204, an evaporator 214, a first shut-off valve 222, and a second shut-off valve 212. The compressor 208, the condenser 204, the evaporator 214, and the first 222 and second 212 shut-off valves are fluidly connected by refrigerant lines 202 (e.g., refrigerant line 202-1, refrigerant line 202-2, etc.) to form a refrigerant circuit.

In some embodiments, the compressor 208 is powered by an engine of a vehicle. In some embodiments, the compressor 208 is powered by an electrical power source 206, such as a battery, or a power line.

The condenser 204 is configured to condense refrigerant compressed by the compressor 208. As depicted in FIG. 2, the condenser 204 is downstream of the compressor 208. As used herein, the term “downstream” refers to a position along a refrigerant line in the direction of the flow of the refrigerant. Similarly, the term “upstream” refers to a position along a refrigerant line opposite to the direction of the refrigerant flow. For example, FIG. 2 illustrates the condenser 204 as downstream of the compressor 208 and upstream of the evaporator 214, where the refrigerant flow direction is indicated by arrows on refrigerant lines 202.

As depicted in FIG. 2, the evaporator 214 is downstream of the condenser 204 and fluidly connected to the condenser 204 by refrigerant lines 202-1 and 202-4. The evaporator 214 is configured to evaporate the condensed refrigerant. The first shut-off valve 222 is installed at the refrigerant inlet of the evaporator 214, and the second shut-off valve 212 is installed at the refrigerant outlet of the evaporator 214.

As used herein, the term “refrigerant inlet” refers to an inlet of a corresponding evaporator and a portion of a refrigerant line upstream of the corresponding evaporator. As used herein, the term “refrigerant outlet” refers to an outlet of a corresponding evaporator and a portion of a refrigerant line downstream of the corresponding evaporator. For example, refrigerant inlet of the evaporator 214 refers to the inlet of the evaporator 214 and a portion of the refrigerant line 202-4 upstream of the evaporator 214. Refrigerant outlet of the evaporator 214 refers to the outlet of the evaporator 214 and a portion of the refrigerant line 202-3 downstream of the evaporator 214.

In some embodiments, the evaporator 214 is in thermal communication with an enclosed space, such as space 102 of FIG. 1, to cool the enclosed space. As used herein, the term “in thermal communication” refers to one or more of the following: (i) the respective evaporator is mounted within a corresponding space to exchange heat with that space or with the air in that space; and (ii) the respective evaporator is coupled with a device (e.g., heat exchanger or air blower) which introduces conditioned air into that space.

As depicted in FIG. 2, in some embodiments, the HVAC system 200 includes a sensor 218 and a controller 224 electrically coupled to the sensor 218. In some embodiments, the sensor 218 is the same as at least one of the sensors 138 of FIG. 1. In some embodiments, the controller 224 is the intelligent power generation management controller described in U.S. Patent App. Pub. No. 2007/0131408, and U.S. Pat. Nos. 7,591,143 and 8,453,722, all of which are expressly incorporated by reference in their entirety.

The sensor 218 is configured to perform one or more of the following: (i) measure the temperature of the evaporator 214; and (ii) measure the airflow passing over the evaporator 214. When the measured temperature is lower than a predetermined temperature, or the measured airflow passing over the evaporator 214 is less than a predetermined volume (e.g., 75 CFM), or both, the controller 224 automatically closes or sends instruction to close the first 222 and second 212 shut-off valves. When the measured temperature exceeds the predetermined temperature or when the measured airflow passing over the evaporator 214 is equal to or greater than the predetermined volume, the controller 224 automatically opens or sends instruction to open the first 222 and second 212 shut-off valves. In some embodiments, the controller 224 is electrically coupled to one or more other components in the HVAC system. For example, in one embodiment, the controller 224 is electrically coupled to the compressor 208 to automatically control the operation of the compressor in accordance with ambient temperature, the cooling demand of the space, or other parameters.

In some embodiments, the HVAC system 200 of the present invention includes a control valve to selectively restrict or permit flow of the refrigerant to the compressors. As an example, FIG. 2 illustrates the HVAC system 200 having a flow control valve 210. The flow control valve 210 is disposed at the refrigerant line 202-3 upstream of the compressor 208 and configured to selectively restrict or permit flow of the refrigerant to the compressor 208. In some embodiments, the operation of the flow control valve 210 is automatically controlled by the controller 224.

In some embodiments, the HVAC system 200 of the present invention includes a metering device to control flow of the refrigerant into the evaporator 214. As an example, FIG. 2 illustrates the HVAC system 200 having a metering device 220. The metering device 220 is disposed at the refrigerant line 202-4 between the first shut off valve 222 and the evaporator 214 and configured for controlling flow rate of the refrigerant into the evaporator 214. In some embodiments, the metering device 220 is a thermal expansion valve. In some embodiments, the operation of the metering device 220 is automatically controlled by the controller 224.

In some embodiments, the HVAC system 200 of the present invention includes a receiver/drier 228. The receiver/drier 228 is disposed at the refrigerant line 202-1, 202-4 between the condenser 204 and the evaporator 214. The receiver/drier 228 is configured to temporarily store the refrigerant, absorb moisture from the refrigerant, or both.

In some embodiments, the HVAC system 200 of the present invention includes one or more blowers and/or one or more fans to enhance the performance of some components in the HVAC system 200. As an example, FIG. 2 illustrates the HVAC system 200 having a blower 216 and a fan 226. The blower 216 is positioned proximate the first evaporator 214 and configured to blow air over the evaporator 214. The air is cooled when passed over the evaporator 214 and can be introduced into a space for cooling purpose. The fan 226 is positioned proximate the condenser 204 and configured to blow air over the condenser 204 to cool it. When passing over the condenser 204, the air extracts the heat away from the condenser 204 and thus enhances the performance of the condenser 204.

FIG. 3 is a block diagram illustrating a controller 300, such as controller 136 of FIG. 1 or the controller 248 of FIG. 2, in accordance with some embodiments of the present invention. In some embodiments, the controller 300 includes one or more central processing units (CPUs) 302, one or more network communication interfaces 316, memory 320, and one or more communication buses 344 for interconnecting these components. In some embodiments, the controller 300 includes a user interface 310, which may include a display 312 or input device(s) 314.

In accordance with some embodiments, the controller 300 is coupled to one or more sensors 304, such as the one or more sensors 138 of FIG. 1 or the sensor 218 of FIG. 2. In some embodiments, the controller 300 is coupled to a baffle 306, such as the baffle 126 of FIG. 1. In some embodiments, the controller 300 is coupled to a humidity controller 308, which may include a humidifier and/or dehumidifier, such as the humidifier/dehumidifier 134 of FIG. 1. In some embodiments, the controller 300 is coupled to a recirculation system 311, such as the recirculation system 118 of FIG. 1. In some embodiments, the controller 300 is coupled to an HVAC system, such as the HVAC system 106 of FIG. 1 or the HVAC system 200 of FIG. 2.

The network communication interface 316 includes, for example, hardware capable of data communications using any of a variety of custom or standard wireless protocols (e.g., IEEE 802.15.4, Wi-Fi, ZigBee, 6LoWPAN, Thread, Z-Wave, Bluetooth Smart, ISA100.11a, WirelessHART, MiWi, etc.) and/or any of a variety of custom or standard wired protocols (e.g., Ethernet, HomePlug, etc.), or any other suitable communication protocol, including communication protocols not yet developed as of the filing date of this document.

In some embodiments, the memory 320 includes high-speed random-access memory, such as DRAM, SRAM, DDR SRAM, or other random-access solid-state memory devices. In some embodiments, the memory 320 includes non-volatile memory, such as one or more magnetic disk storage devices, one or more optical disk storage devices, one or more flash memory devices, or one or more other non-volatile solid state storage devices. In some embodiments, the memory 320 includes a non-transitory, computer-readable storage medium. In some embodiments, the memory 320, or the non-transitory, computer-readable storage medium of memory 320, stores the following programs, modules, and data structures, or a subset or superset thereof: (1) an operating system 322, for handling various system services and for performing hardware-dependent tasks; (2) a communications module 324, for coupling to and communicating with other network devices via one or more networks (e.g., in conjunction with the network communication interface 316); (3) a user interface module 326, for receiving input from, and displaying information to, a user; (4) a sensor module 328, for monitoring and collecting data from various sensors, such as the one or more sensors 138 of FIG. 1 or the sensor 218 of FIG. 2; (5) a humidity control module 330, for increasing, decreasing, or maintaining the humidity of a space, such as the enclosed space 102 of FIG. 1; and (6) an HVAC system control module 332, for operating the HVAC system 318.

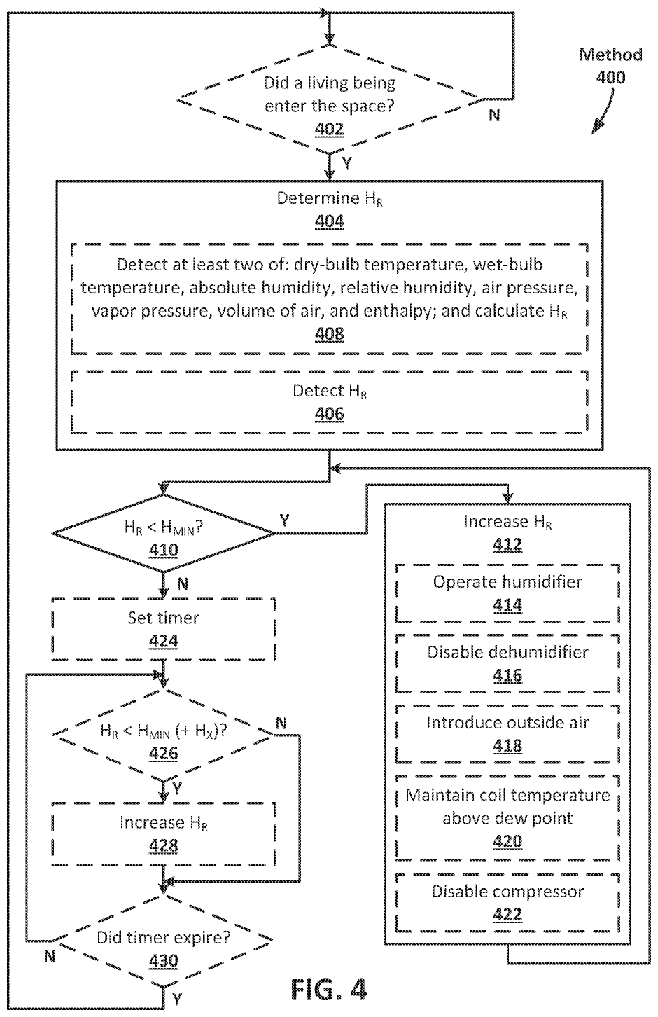

In some embodiments, the sensor module 328 receives data corresponding to measurements from one or more sensors, such as the one or more sensors 138 of FIG. 1 or the sensor 218 of FIG. 2. In some embodiments, the sensor module 328 receives data corresponding to the humidity ratio of an enclosed space, such as the enclosed space 102 of FIG. 1. In some embodiments, the sensor module 328 generates an alert or sets a flag when the humidity ratio of the enclosed space is at, above, or below a threshold, such as the minimum or maximum humidity ratio thresholds described with respect to FIG. 4. In some embodiments, the sensor module 328 sends a signal to the humidity control module 330 when the humidity ratio of the enclosed space is at, above, or below a threshold. In some embodiments, the signal from the sensor module 328 to the humidity control module 330 indicates that the humidity control module 330 should increase, decrease, or maintain the humidity ratio of the enclosed space.

In some embodiments, the sensor module 328 receives data corresponding to measurements from one or more sensors, such as the one or more sensors 138 of FIG. 1 or the sensor 218 of FIG. 2. In some embodiments, the sensor module 328 receives data suggesting a living being might be approaching, entering, or occupying an enclosed space, such as the enclosed space 102 of FIG. 1. For example, in some embodiments, the sensor module 328 receives data from the one or more sensors and uses the data to determine whether a living being approached, entered, or occupied the enclosed space. In some embodiments, the sensor module 328 generates an alert or sets a flag when it determines that a living being approached, entered, or occupied the enclosed space. In some embodiments, the sensor module 328 initiates a method, such as the method 400 of FIG. 4 when it determines that a living being approached, entered, or occupied the enclosed space.

In some embodiments, the humidity control module 330 receives input from the sensor module 328. In some embodiments, the signal from the sensor module 328 includes a request to increase, decrease, or maintain the humidity ratio of an enclosed space, such as the enclosed space 102 of FIG. 1. In some embodiments, the humidity control module 330 increases the humidity ratio of the enclosed space, such as by initiating any of steps 414, 416, 418, 420, and 422 of FIG. 4. In some embodiments, the humidity control module 330 decreases the humidity ratio of the enclosed space, such as by ceasing or reversing any of steps 414, 416, 418, 420, and 422 of FIG. 4. And in some embodiments, the humidity control module 330 maintains the humidity ratio of the enclosed space, such as by intermittently performing any of steps 414, 416, 418, 420, and 422 of FIG. 4.

Each of the above-identified elements corresponds to a set of instructions for performing a function described herein. The above identified modules or programs (i.e., sets of instructions) need not be implemented as separate software programs, procedures, or modules, and thus various subsets of these modules may be combined or otherwise rearranged in various embodiments. In some embodiments, the memory 320, optionally, stores a subset of the modules and data structures identified above. In some embodiments, the memory 320 stores additional modules and data structures not described above.

FIG. 4 is a flow chart of a method 400 for reducing virus transmission in an enclosed space, such as the enclosed space 102 of FIG. 1. As with the enclosed space 102 of FIG. 1, in some embodiments, the enclosed space discussed with respect to FIG. 4 is an interior portion of a vehicle, such as the cabin of a truck, an airplane, a ship, or a train. Likewise, in some embodiments, the enclosed space is an interior portion of a building, such as a room in a home, a classroom in a school, an auditorium in a theatre, or the floor of an office building. In some embodiments, the enclosed space 102 is the entire interior of a vehicle (e.g., the cabin, cockpit, etc. of an airplane) or a building (e.g., each room in a home). The virus discussed with respect to FIG. 4 is any known or yet to be discovered virus that is sensitive to humidity (e.g., able to be killed by high or low humidity), including the influenza A, influenza B, and SARS-CoV-2 viruses.

In some embodiments, it is determined (402) whether a living being entered the enclosed space. For example, in some embodiments, one or more sensors, such as the one or more sensors 138 of FIG. 1 or the sensor(s) 304 of FIG. 3, are used to detect that a living being entered the space. Alternatively, or additionally, in some embodiments, the method includes determining whether a living being is approaching the enclosed space or occupying the enclosed space. For example, in some embodiments, the method includes receiving a signal (e.g., a message, a WiFi connection, a Bluetooth connection, GPS data) from an electronic device (e.g., a smart phone, a laptop, an identification badge) of a user indicating a living being is approaching or occupying the enclosed space. As another example, in some embodiments, the method includes using artificial intelligence (e.g., computer vision) to interpret data collected by a camera to determine whether a living being is approaching or occupying the enclosed space.

Determining first whether a living being is approaching, entering, or occupying the enclosed space is useful for purposes of efficiently decreasing virus transmission. For example, if the enclosed space is empty (i.e., there are no living beings in the enclosed space), the risk of virus transmission between living beings in the enclosed space is null. Similarly, if the enclosed space only has one living being in it, there is no risk of virus transmission between living beings in the enclosed space. However, if for example there is already a living being in the enclosed space and another living being enters the enclosed space, there may be a risk of virus transmission between the two living beings. Accordingly, in some embodiments, it is first determined whether a living being is approaching, entering, or occupying the enclosed space in order to determine there is a risk of virus transmission.

Generally, “living beings” denotes animals (e.g., humans), plants, fungi, protists, and bacteria. However, in some embodiments, the determination regarding whether a living being is approaching, entering, or occupying the enclosed space includes filtering for certain kinds of living beings. For example, in some embodiments, the determination includes determining whether a human being is approaching, entering, or occupying the enclosed space. As another example, in some embodiments, the determination includes filtering for animals (i.e., non-human animals). As another example, in some embodiments, the determination includes determining whether a plant is approaching, entering, or occupying the enclosed space. In some embodiments, determining whether a certain kind of living being is approaching, entering, or occupying the enclosed space includes using artificial intelligence (e.g., computer vision) to filter for the certain kind of living being.

In some embodiments, if it is determined (402—N) that a living being is not approaching, entering, or occupying the enclosed space, the method loops to continue determining (402) whether a living being is approaching, entering, or occupying the enclosed space. In other words, the determination (402) is repeated until it is determined (402—Y) a living being is approaching, entering, or occupying the enclosed space. In some embodiments, the method includes continuing to determine (402) whether a living being is approaching, entering, or occupying the enclosed space (e.g., concurrently with other steps of method 400) even after determining a living being is approaching, entering, or occupying the space.

Once it is determined (402—Y) that a living being is approaching, has entered, or is in the enclosed space (or in certain embodiments that do not include first determining whether a living being entered the enclosed space), the method includes determining (404) the humidity ratio (H_{R}) of the enclosed space. In other embodiments, the method includes determining the relative humidity of the space, the absolute humidity of the space, or the humidity ratio of the space.

In other embodiments, other humidity measures, such as relative humidity, absolute humidity, or specific humidity are used instead of the humidity ratio.

As noted above, for some viruses, humidity is related to the virus' transmission rates. For example, influenza transmission rates are known to decrease in high-humidity environments. Accordingly, in some embodiments, determining the humidity ratio (H_{R}) of the enclosed space is useful in determining whether the humidity ought to be increased to decrease a transmission rate in the enclosed space of a virus. Similarly, determining other humidity measures, such as the aforementioned absolute humidity, relative humidity, humidity ratio, and so on, may also be useful in establishing a baseline predicted virus transmission rate and determining whether the humidity ought to be increased.

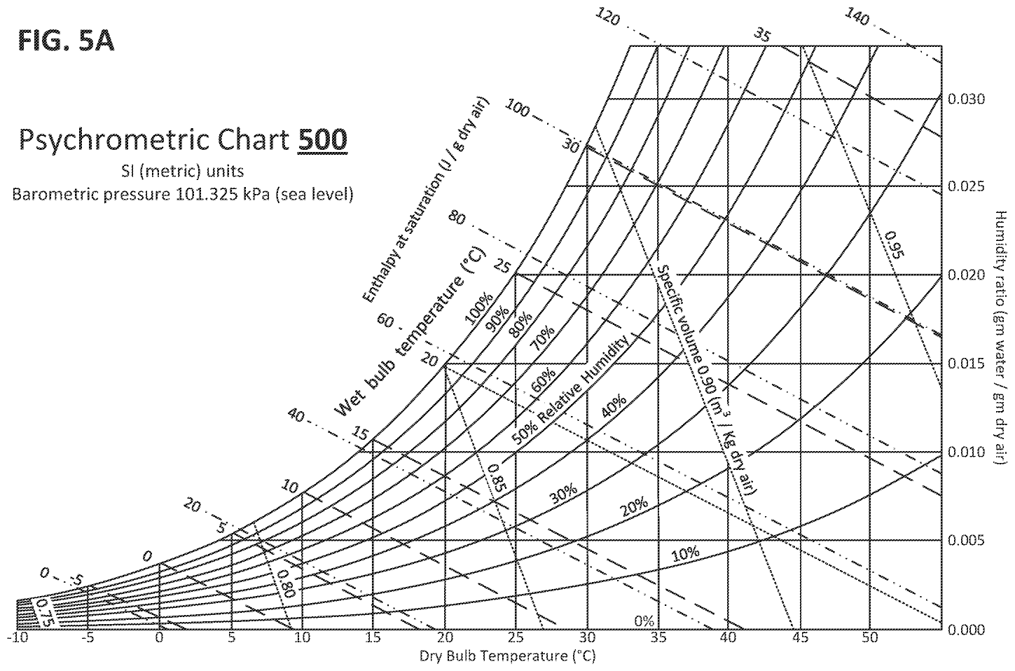

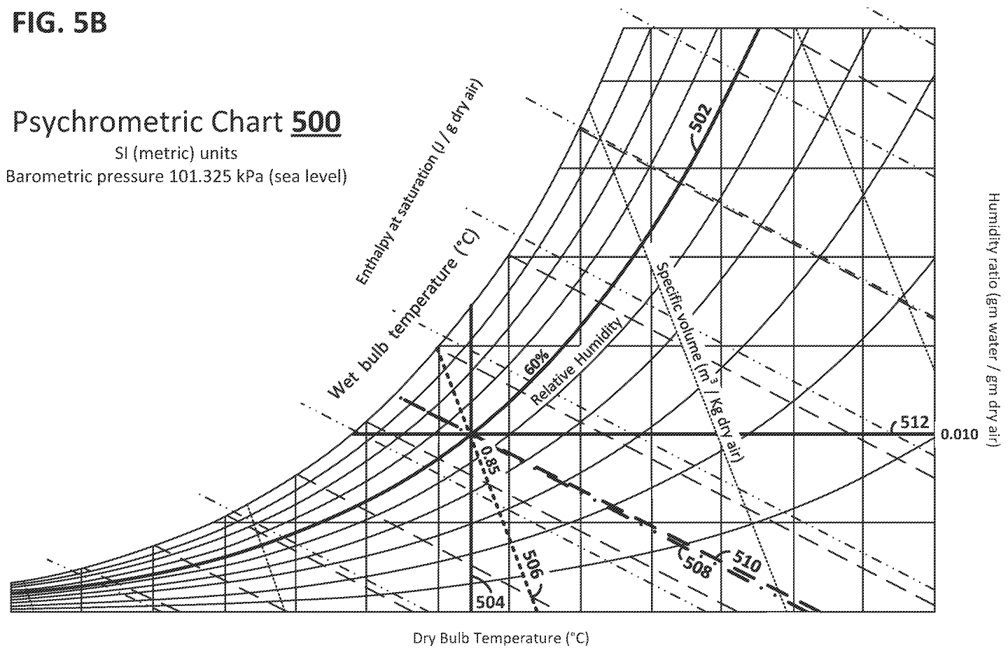

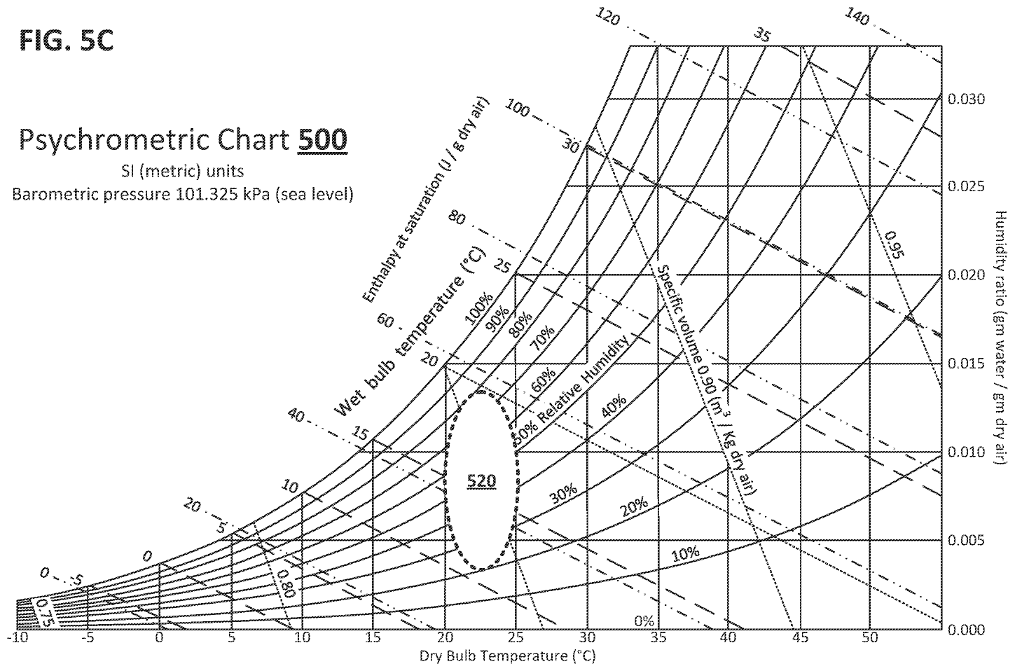

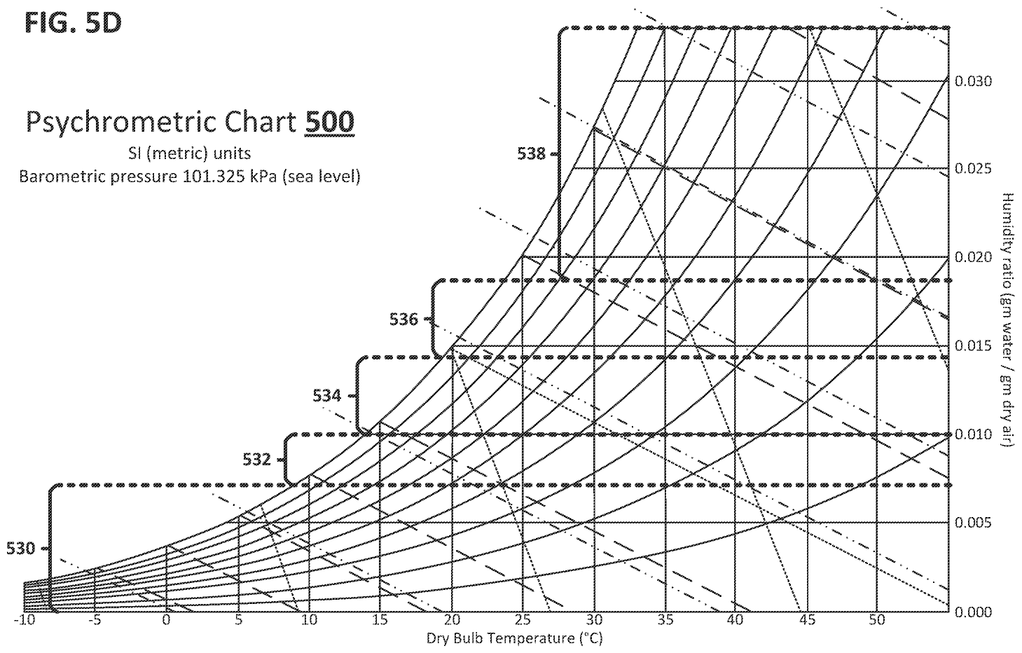

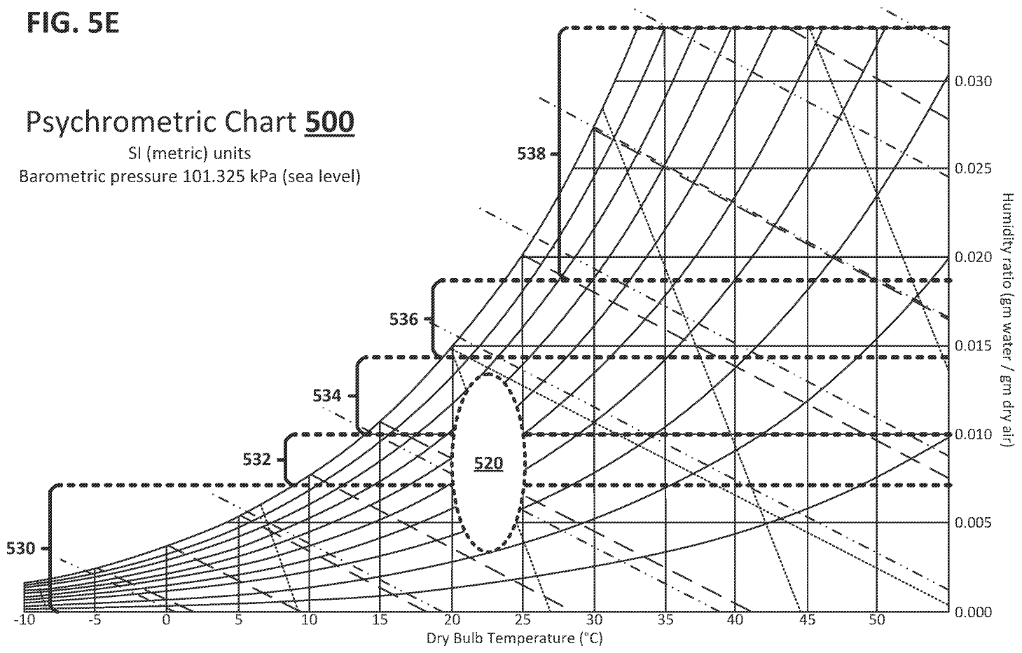

In some embodiments, determining the humidity ratio (H_{R}) of the enclosed space includes detecting (406) the humidity ratio with a sensor capable of detecting humidity ratio. In some embodiments, the determination includes detecting (408) other metrics from which the humidity ratio can be calculated. For example, in some embodiments, the other metrics include at least two of dry-bulb temperature, wet-bulb temperature, absolute humidity, relative humidity, air pressure, vapor pressure, volume of air, enthalpy, mass of the water in a moist air sample, and the total mass of the moist air sample. Those skilled in the art will understand how to calculate humidity ratio from these metrics. For example, in some embodiments, the humidity ratio is calculated using a psychrometric chart. For a brief discussion of psychrometric charts as they relate to embodiments of the present invention, see FIGS. 5A-5E.

As another example, in some embodiments, the humidity ratio is calculated using the following formula:

$$W = \frac{{m}_{w}}{{m}_{a}} = 0.62198 \frac{{p}_{w}}{{p}_{a} - {p}_{w}}$$

where W is the humidity ratio, m_{w} is the mass of the water vapor in a moist air sample, mₐ is the mass of the air in the moist air sample, p_{w} is the partial pressure of the water vapor in the moist air sample, and pₐ is the atmospheric pressure of the moist air sample.

In some embodiments, the method includes determining (410) whether the humidity ratio of the enclosed space is less than a minimum humidity ratio threshold (H_{MIN}). In some embodiments, the minimum humidity ratio threshold (H_{MIN}) is based on a minimum humidity ratio at which the half-life of a virus is decreased by a predetermined amount. In some embodiments, the minimum humidity ratio threshold (H_{MIN}) is based on a minimum humidity ratio at which the transmission rate of a virus is decreased by a predetermined amount.

In some embodiments, the minimum humidity ratio threshold (H_{MIN}) is within a comfort zone, such as the ASHRAE Standard 55 comfort zone, the EN 15251 comfort zone, or the Givoni-Milne Bioclimatic Chart comfort zone. In other words, in some embodiments, the minimum humidity ratio threshold (H_{MIN}) falls within a zone defining temperatures and humidities comfortable (e.g., not too hot, too cold, too humid, or too dry) for a human being (e.g., 68 degrees F. with 50% RH calculates to a humidity ratio of approx. 0.0073 (lb moisture)/(lb dry air) and a high transmission rate potential).

Alternatively, in some embodiments, the minimum humidity ratio threshold (H_{MIN}) is slightly outside of the comfort zone (e.g., 1%, 2%, or 5% outside of the comfort zone). For example, if lowering the transmission rate of a virus is especially important, the humidity ratio might be raised outside of the comfort zone. In some embodiments, this may mean the temperature and/or humidity of the enclosed space is slightly uncomfortable for a limited amount of time in order to decrease the likelihood of the virus transmitting from one living being in the enclosed space to another living being in the enclosed space.

In some embodiments, if it is determined (410—Y) that the humidity ratio of the enclosed space is less than the minimum humidity ratio threshold (H_{MIN}), the humidity ratio is increased (412). For example, in some embodiments, increasing the humidity ratio includes one or more of the following: operating (414) a humidifier; disabling (416) a dehumidifier; introducing (418) air from outside the enclosed space into the enclosed space; maintaining (420) the temperature of a coil above a dew point; and disabling (422) a compressor. In some embodiments, if it is determined (410—Y) that the humidity ratio of the enclosed space is less than the minimum humidity ratio threshold (H_{MIN}), the method loops to continue increasing (412) the humidity ratio of the enclosed space until it is determined (410—N) that the humidity ratio is not less than the minimum humidity ratio threshold (H_{MIN}).

In some embodiments, once it is determined (410—N) the humidity ratio of the enclosed space is not less than (i.e., is greater than and/or equal to) the minimum humidity ratio threshold (H_{MIN}), a timer is set (424) a timer. In some embodiments, the timer is a countdown timer. In some embodiments, the duration of the timer is based on the half-life of a virus at the humidity ratio associated with the minimum humidity ratio threshold. In some embodiments, the duration of the timer is based on the transmission rate of a virus at the humidity ratio associated with the minimum humidity ratio threshold.

In some embodiments, once it is determined (410—N) the humidity ratio of the enclosed space is not less than (i.e., is greater than and/or equal to) the minimum humidity ratio threshold (H_{MIN}), the humidity ratio is increased (not pictured) until it reaches or exceeds a maximum humidity ratio threshold. In some embodiments, after the humidity ratio reaches the maximum humidity ratio threshold, the humidity ratio is then maintained for a predetermined amount of time. In other embodiments, after the humidity ratio reaches the maximum humidity ratio threshold, the method again loops (402) to determine whether a living being is approaching, entering, or occupying the enclosed space.

In some embodiments, it is determined (426) whether the humidity ratio of the enclosed space is less than the minimum humidity ratio threshold. In other embodiments, it is determined (426) whether the humidity ratio is less than the minimum humidity ratio threshold, plus some buffer amount (H_{X}). Further, in some embodiments, it is determined (not pictured) whether the humidity ratio is greater than the maximum humidity ratio threshold. If it is determined the humidity ratio is greater than the maximum humidity ratio threshold, the humidity ratio is then decreased.

In some embodiments, a proportional-integral-derivative (PID) controller is used to maintain (not pictured) the humidity ratio above the minimum humidity ratio threshold (H_{MIN}) for a predetermined amount of time. In some embodiments, the PID controller is also used to maintain (not pictured) the humidity ratio below a maximum humidity ratio threshold for the predetermined amount of time. In some embodiments, the predetermined amount of time is based on a half-life of a virus, the minimum humidity ratio threshold (H_{MIN}), the maximum humidity ratio threshold, the size of the enclosed space, the prevalence of the virus, the number of living beings in the enclosed space, and/or the proximity of the living beings to each other.

Maintaining the humidity ratio (H_{R}) of the enclosed space within a comfort zone is important for keeping the climate of the enclosed space comfortable for the living beings occupying it. Keeping the humidity ratio (H_{R}) within the comfort zone also helps to avoid issues associated with especially humid climates, such as mold. However, in some embodiments, for example during a global pandemic when decreasing virus transmission rates is paramount, the humidity ratio (H_{R}) of the enclosed space may be increased (or decreased, if and where applicable) beyond the bounds of the comfort zone in order to further decrease virus transmission rates.

In some embodiments, if it is determined (426—Y) the humidity ratio is less than the minimum humidity ratio threshold and/or the sum of the minimum humidity ratio threshold (H_{MIN}) and the buffer amount (H_{X}), then the humidity ratio is increased (428). In some embodiments, increasing the humidity ratio includes, for example, any of the steps recited above with respect to step 412.

In some embodiments, if it is determined (426—N) the humidity ratio is not less than (i.e., is greater than or equal to) the minimum humidity ratio threshold and/or the sum of the minimum humidity ratio threshold (H_{MIN}) and the buffer amount (H_{X}), the method then includes determining (430) whether the timer expired.

If it is determined (430—N) the timer did not expire, in some embodiments, the method then includes re-determining (426) whether the humidity ratio is less than the minimum humidity ratio threshold. Likewise, in other embodiments, if it is determined (430—N) the timer is not expired, the method then includes re-determining (426) whether the humidity ratio is less than the sum of minimum humidity ratio threshold and the buffer amount.

In some embodiments, if it is instead determined (430—Y) that the timer expired, it is then determined (402) whether a living being approached, entered, or occupied the enclosed space. Alternatively, in some embodiments, after determining (430—Y) that the timer expired, the humidity ratio of the enclosed space is then determined (404).

FIGS. 5A-5E are example psychrometric charts, some of which include notations that demonstrate certain aspects of the described embodiments. A psychrometric chart is a graph of thermodynamic parameters of air. For example, the psychrometric chart 500 shown in FIG. 5A depicts relative humidity, dry bulb temperature, specific volume, specific enthalpy, wet-bulb temperature, humidity ratio, and dew point temperature. Those skilled in the art will understand how to read a psychrometric chart, such as psychrometric chart 500. Nonetheless, FIG. 5B includes a brief explanation of the interrelation of the various parameters represented in the psychrometric chart 500.

FIG. 5B includes lines representing six parameters depicted by the psychrometric chart 500. First, line 502 denotes a relative humidity. Second, line 504 denotes a dry bulb temperature. Third, line 506 denotes a specific volume. Fourth, line 508 denotes a specific enthalpy. Fifth, line 510 denotes a wet bulb temperature. And sixth, line 512 denotes a humidity ratio.

The intersection of lines 502, 504, 506, 508, 510, and 512 illustrates the utility of the psychrometric chart 500: It allows for the calculation of some metrics based on knowledge of at least two other metrics. For example, based on a measured relative humidity of 60% and a measured dry bulb temperature of 22° C., someone could use the psychrometric chart 500 to determine the humidity ratio. They would first find the intersection of the line representing 60% relative humidity (i.e., line 502) and the line representing a dry bulb temperature of 22° C. (i.e., line 504). Then, they would trace the intersection of those two lines to the right (i.e., following line 512) to determine that the humidity ratio was 0.010 grams of water per grams of dry air.

FIG. 5C includes an example comfort zone 520. The comfort zone 520 represents a range of temperatures and humidities that most people would consider comfortable. For example, a dry bulb temperature of 22° C. (71.6° F.) and a relative humidity of 50% would fall within the comfort zone 520. In other words, most people would consider comfortable a room kept at a dry bulb temperature of 22° C. and a relative humidity of 50%—assuming, of course, the depicted comfort zone 520 accurately represents what most people would find comfortable.

The depicted comfort zone 520 is an oval shape that stretches from 20 to 25° C. dry bulb temperature, and from 20 to 80% relative humidity. However, the comfort zone 520 is merely an example comfort zone. For purposes of the present invention, in some embodiments, the comfort zone is a comfort zone other than the depicted comfort zone 520. For example, the comfort zone may be a standard comfort zone, such as the ASHRAE Standard 55 comfort zone, the EN 15251 comfort zone, or the Givoni-Milne Bioclimatic Chart comfort zone.

FIG. 5D includes five transmission zones denoting different degrees of virus transmission. Like the comfort zone 520, the depicted transmission zones 530, 532, 534, 536, and 538 are provided for illustrative purposes only. References elsewhere in the specification to “transmission zones” or the like may refer to zones with bounds different than those depicted in FIG. 5D. Further, in some embodiments, transmission zones will change based on the corresponding virus. For example, the zero-transmission zone 538 may only exist at extremely high humidities for a first virus; however, the zero-transmission zone 538 may exist at mid- to high-level humidities for a second virus.

That said, transmission zones 530, 532, 534, 536, and 538 are divided by horizontal lines corresponding to various humidity ratios. As noted elsewhere in the specification, the transmission rates of certain viruses decrease with an increase in humidity. Accordingly, the five transmission zones depicted in FIG. 5D represent various degrees of risk for transmission of a virus. For example, transmission zone 530 represents a high-risk transmission zone. At humidities in transmission zone 530, transmission rates for a virus are extremely high (e.g., 68 F with 25% RH, zone 1, Correlating to a humidity ratio of 0.0036 (lb moisture)/(lb dry air) and a 80-100% probability rate of transmission). Transmission zone 532 represents a medium-risk transmission zone. Transmission rates in this zone are somewhat lower than rates in zone 530. Transmission zone 534 represents a stable zone. Transmission zone 536 represents a low-risk zone. And transmission zone 538 represents a zero-transmission zone. The risk of virus transmission in this zone is zero—or very close to it.

FIG. 5E includes the comfort zone 520 of FIG. 5C, as well as the transmission zones 530, 532, 534, 536, and 538 of FIG. 5D. As depicted in FIG. 5E, the comfort zone 520 intersects multiple transmission zones, including transmission zone 530, transmission zone 532, and transmission zone 534. As noted with respect to FIG. 5C, comfortable climates are defined by the bounds of the comfort zone 520. And, as noted above with respect to FIG. 5D, the risk of virus transmission decreases in higher zones. Accordingly, the present invention is directed to maintaining a high humidity ratio (i.e., staying in the lowest-risk transmission zone possible) while also remaining in the comfort zone 520.

### Example Aspects

A few example aspects will now be briefly described.
(A1) In accordance with some embodiments, method for reducing virus transmission in a space is provided. The method includes providing a minimum humidity ratio threshold at which transmission of a particular virus is decreased. The method further includes determining a humidity ratio of an enclosed space suitable for receiving a living being, and in response to determining that the humidity ratio is less than the minimum humidity ratio threshold, increasing the humidity ratio until the humidity ratio in the enclosed space is at least equal to the minimum humidity ratio threshold.
(A2) In some embodiments of A1, the minimum humidity ratio threshold comprises a minimum humidity ratio at which a half-life of a virus is decreased.
(A3) In some embodiments of any of A1-A2, the minimum humidity ratio threshold is within a comfort zone.
(A4) In some embodiments of any of A1-A3, the comfort zone comprises one of: an ASHRAE Standard 55 comfort zone; an EN 15251 comfort zone; or a Givoni-Milne Bioclimatic Chart comfort zone.
(A5) In some embodiments of any of A1-A4, the virus is influenza A.
(A6) In some embodiments of A5, the minimum humidity ratio threshold is 0.01 lb/lb.
(A7) In some embodiments of any of A1-A6, the virus is SARS-CoV-2.
(A8) In some embodiments A7, the minimum humidity ratio threshold is 0.01 lb/lb.
(A9) In some embodiments of any of A1-A8, determining the humidity ratio includes detecting the humidity ratio with a sensor.
(A10) In some embodiments of any of A1-A9, determining the humidity ratio includes detecting at least two of the following metrics: a dry-bulb temperature of the space, a wet-bulb temperature of the space, an absolute humidity of the space, a relative humidity of the space, an air pressure of the space, a vapor pressure of the space, a volume of air in the space, and an enthalpy of the air in the space; and calculating the humidity ratio based on the at least two detected metrics.
(A11) In some embodiments of any of A1-A10, the enclosed space comprises an interior of a vehicle.
(A12) In some embodiments of any of A1-A10, the enclosed space comprises a room.
(A13) In some embodiments of any of A1-A12, the living being is a human being.
(A14) In some embodiments of any of A1-A13, increasing the humidity ratio includes at least one of: operating a humidifier; disabling a dehumidifier; introducing air outside the space into the space; maintaining a temperature of a coil above a dew point of the space; and disabling a compressor.
(A15) In some embodiments of any of A1-A14, the method further includes, before increasing the humidity ratio, detecting a living being entering the space.
(A16) In some embodiments of any of A1-A15, the method further includes maintaining, after determining that the humidity ratio is at least equal to the minimum humidity ratio threshold, the humidity ratio.
(A17) In some embodiments of any of A1-A16, the method further includes maintaining, after determining the humidity ratio is at least equal to a maximum humidity ratio threshold, the humidity ratio between the minimum humidity ratio threshold and the maximum humidity ratio threshold.
(A18) In some embodiments of A17, the maximum humidity ratio threshold is within a comfort zone.
(A19) In some embodiments of any of A16-A18, maintaining the humidity ratio comprises maintaining the humidity ratio for a predetermined amount of time.
(A20) In some embodiments of A19, the predetermined amount of time is based on a half-life of a virus at the minimum humidity ratio threshold.
(A21) In some embodiments of any of A1-A20, the method further includes decreasing, after determining the humidity ratio is greater than a maximum humidity ratio threshold, the humidity ratio.
(B1) In accordance with some embodiments, system for reducing virus transmission in a space is provided. The system (e.g., system of FIGS. 1 and 2) includes one or more sensors and a controller electrically coupled to the one or more sensors. The controller is configured to perform the method of any of A1-A21.
(C1) In accordance with some embodiments, a non-transitory, computer-readable medium comprising instructions that, when executed by one or more processors of a system (e.g., system of FIGS. 1 and 2), cause the one or more processors to perform the method of any of A1-A21.

It will be understood that, although the terms “first,” “second,” etc. may be used herein to describe various elements, these elements should not be limited by these terms. These terms are only used to distinguish one element from another.

The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” can be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” can be construed to mean “upon determining” or “in response to determining” or “in accordance with a determination” or “upon detecting” or “in response to detecting” that the stated condition precedent is true, depending on the context.

What is claimed is:

1. A method for reducing virus transmission in a space, comprising:
providing a minimum humidity ratio threshold at which transmission of a particular virus is decreased;
determining a humidity ratio of an enclosed space suitable for receiving a living being; and
in response to determining that the humidity ratio is less than the minimum humidity ratio threshold, increasing the humidity ratio until the humidity ratio in the enclosed space is at least equal to the minimum humidity ratio threshold.

2. The method of claim 1, wherein the minimum humidity ratio threshold comprises a minimum humidity ratio at which a half-life of a virus is decreased.

3. The method of claim 1, wherein the minimum humidity ratio threshold is within a comfort zone.

4. The method of claim 3, wherein the comfort zone comprises one of: an ASHRAE Standard comfort zone; an EN 15251 comfort zone; or a Givoni-Milne Bioclimatic Chart comfort zone.

5. The method of claim 1, wherein the virus is influenza A and/or SARS-CoV-2.

6. The method of claim 5, wherein the minimum humidity ratio threshold is 0.01 lb/lb.

7. The method of claim 1, wherein determining the humidity ratio comprises detecting the humidity ratio with a sensor.

8. The method of claim 1, wherein determining the humidity ratio comprises:
detecting at least two of the following metrics: a dry-bulb temperature of the space, a wet-bulb temperature of the space, an absolute humidity of the space, a relative humidity of the space, an air pressure of the space, a vapor pressure of the space, a volume of air in the space, and an enthalpy of the air in the space; and
calculating the humidity ratio based on the at least two detected metrics.

9. The method of claim 1, wherein the enclosed space comprises an interior of a vehicle.

10. The method of claim 1, wherein the enclosed space comprises a room.

11. The method of claim 1, wherein increasing the humidity ratio comprises at least one of: operating a humidifier; disabling a dehumidifier; introducing air outside the space into the space; maintaining a temperature of a coil above a dew point of the space; and disabling a compressor.

12. The method of claim 1, further comprising: before increasing the humidity ratio, detecting a living being entering the space.

13. The method of claim 1, further comprising: maintaining, after determining that the humidity ratio is at least equal to the minimum humidity ratio threshold, the humidity ratio.

14. The method of claim 1, further comprising: maintaining, after determining the humidity ratio is at least equal to a maximum humidity ratio threshold, the humidity ratio between the minimum humidity ratio threshold and the maximum humidity ratio threshold.

15. The method of claim 14, wherein the maximum humidity ratio threshold is within a comfort zone.

16. The method of claim 14, wherein maintaining the humidity ratio comprises maintaining the humidity ratio for a predetermined amount of time.

17. The method of claim 16, wherein the predetermined amount of time is based on a half-life of a virus at the minimum humidity ratio threshold.

18. The method of claim 1, further comprising: decreasing, after determining the humidity ratio is greater than a maximum humidity ratio threshold, the humidity ratio.

19. A system for reducing virus transmission in a space, comprising:
one or more sensors;
a controller electrically coupled to the one or more sensors, wherein the controller is configured to:
provide a minimum humidity ratio threshold at which transmission of a particular virus is decreased;
determine a humidity ratio of an enclosed space suitable for receiving a living being; and
in response to determining that the humidity ratio is less than the minimum humidity ratio threshold, increase the humidity ratio until the humidity ratio in the enclosed space is at least equal to the minimum humidity ratio threshold.

20. A non-transitory, computer-readable medium comprising instructions that, when executed by one or more processors of a system for reducing virus transmission in a space, cause the one or more processors to:
provide a minimum humidity ratio threshold at which transmission of a particular virus is decreased;
determine a humidity ratio of an enclosed space suitable for receiving a living being; and
in response to determining that the humidity ratio is less than the minimum humidity ratio threshold, increase the humidity ratio until the humidity ratio in the enclosed space is at least equal to the minimum humidity ratio threshold.
